(19) United States
(12) Patent Application Publication
(10) Pub. No.: US 2008/0177234 A1
(43) Pub. Date: Jul. 24, 2008

(54) Safety subcutaneous infusion set

(76) Inventors: Candace Keaton, Pasadena, MD (US); Alan Reid, Keene, NH (US)

Correspondence Address: Ober, Kaler, Grimes & Shriver, 120 East Baltimore Street, Baltimore, MD, 21202-1643, US

(21) Appl. No.: 11/986,547

(22) Filed: Nov. 21, 2007

Related U.S. Application Data
(60) Provisional application No. 60/860,314, filed on Nov. 21, 2006.

(51) Int. Cl. A61M 25/02

(52) U.S. Cl. 604/177

(57) ABSTRACT

A safety butterfly infusion set comprising a catheter, a length of flexible tubing in fluid communication with the catheter, and a butterfly anchor formed with a central body having a notch formed therein and leading sideward into the body for centering and seating a delivery tube in a press-fit manner. The butterfly anchor also has hinges on opposing sides of the body that support a pair of opposing foldable wings. A locking post protrudes from the distal end of one wing and when the wings are folded together the locking post may be inserted into a hole at the distal tip of the other wing for releasably locking the wings together. Thus, the wings can be conveniently folded over the catheter and latched in place to protect the user from pricking after use. On the other hand, when the wings are unfolded for use they lay perfectly flat against a patient's skin, thereby allowing adhesion of a strip of dermal tape such as Tegaderm™ overtop to anchor the catheter in place and promote needle stability.

### CROSS-REFERENCE TO RELATED APPLICATIONS

The present application derives priority from U.S. Provisional Patent Application No. 60/860,314 for “SAFETY SUBCUTANEOUS INFUSION SET”; Filed: Nov. 21, 2006.

### BACKGROUND OF THE INVENTION

1. Field of the Invention

The present invention relates to subcutaneous (IV) systems and equipment and, more particularly, to a subcutaneous infusion set with a winged anchor in which the wings fold over the needle/catheter and latch in place to protect it after use, and yet the wings when unfolded lie flat against the patient's skin to allow a dermal adhesion strip to be fastened overtop to anchor the wings to the skin to promote needle stability.

2. Description of the Background

Subcutaneous or IV systems deliver fluid, medications, blood products and parenteral nutrition to patients. Most IV machines operate by gravity, or by an infusion pump (a pump, usually peristaltic, used to control the flow through the IV tube), or by a syringe pump (a pump, usually motor driven using an actuator assembly pushing the plunger of a syringe thus infusing into a patient via a subcutaneous infusion set connected to the syringe). A conventional IV administration system includes an IV machine with injectable solution bag, opening to a drip chamber, connected by flexible tubing to a roller clamp and on to a subcutaneous infusion set for administration to a patient. These subcutaneous infusion sets typically include a needle/catheter (90 degree angle) attached microbore tubing, and some means of anchoring the needle/catheter to the skin of the patient. This anchoring means typically comprises a winged member that keeps the needle captive and protruding subcutaneously at 90 degrees. Unlike the commonly referred to “butterfly” infusion sets that are used for intravenous infusions, the 90 degree needle of a subcutaneous set enters the skin so it reaches down to the subcutaneous tissue. The are adhered to the skin to promote needle stability. The wings are most typically adhered by applying a strip of patient dressing such as Tegaderm™ over the wings and attaching it to the skin. Tegaderm™ is manufactured by 3M Corp. and consists of a thin polyurethane membrane coated with a layer of an acrylic adhesive. The dressing, once in position, provides an effective anchor as well as a barrier to external contamination. Due to the raised public concern over infectious diseases such as AIDS and the like there are now a variety of “safety” subcutaneous infusion sets designed to provide protection for nurse practitioners and other care providers when handling the needles. These safety sets typically shroud the tip of the needle in some way after it is removed from the patient. For example, U.S. Pat. No. 4,941,881 to Masters et al. issued Jul. 17, 1990 shows a safety IV infusion set with a sheath slidably disposed on the tube and adapted to be slid over the needle to cover the needle after use. This particular sheath is adapted for use with the above-described butterfly IV infusion sets.

U.S. Pat. No. 5,330,438 to Gollobin et al. issued Jul. 19, 1994 shows a protecting sheath for an IV infusion set with outwardly projecting flexible wings adapted to be slid over the needle to cover the needle after use. U.S. Pat. No. 6,001,083 to Wilner issued Dec. 14, 1999 shows protective wings that slide closed upon themselves and the needle to protect against inadvertent contact.

U.S. Pat. No. 7,048,718 to Mithiue et al. issued May 23, 2006 likewise shows a winged injection needle with a slidable hub that encloses the needle. Although these sliding-sheath type infusion sets make strides toward a safer operating environment it has been found that significant manual dexterity is necessary to slide the sheath, and users are prone to grasping the needle in order to attain the needed leverage. This of course defeats the purpose. It is more sensible to use the wings themselves as a shroud, and to make the wings foldable over the needle to protect it after use.

U.S. Pat. No. 5,147,319 to Ishikawa et al. issued Sep. 15, 1992 shows such a device, with wings that fold over the needle to allow the needle to be safely exposed/covered, thereby reducing the possibility of puncturing the user's hands. A user holds the wings 5a, 5b and folds the wings towards the needle 2 in order to form the sheath 6 to cover the needle. Male and female parts 10a, 10b couple the wings together to maintain the formation of the sheath or the bend of the wings, as shown in FIG. 3. Unfortunately, this particular configuration of male and female coupling parts 10a, 10b is fairly obtrusive. The wings do not lay flat against the patient's skin and this inhibits the use of a Tegaderm™ strip to anchor the wings to the skin to promote needle stability.

It would be much more advantageous to provide an infusion set with a winged anchor in which the wings fold over the needle and latch in place to protect it after use, and yet the wings when unfolded lie flat against the patient's skin to allow a Tegaderm™ strip to be fastened overtop to anchor the wings to the skin to promote needle stability.

### SUMMARY OF THE INVENTION

It is therefore an object of the present invention to provide a safety subcutaneous infusion set that is easy to use, with wings that fold over the catheter and positively (yet removably) latch in place to increase protection against inadvertent pricking after use.

It is another object to provide a safety subcutaneous infusion set with latching wings that lie perfectly flat against a patient's skin while unfolded and in use, thereby allowing adhesion of a strip of dermal tape such as Tegaderm™ overtop the wings to anchor the catheter in place and promote needle stability.

In accordance with the foregoing objects, the present invention provides a subcutaeous infusion set comprising a 90 degree catheter, a length of flexible tubing in fluid communication with the catheter, and a butterfly anchor including a central body with a defined notch formed therein for seating the catheter and tubing. The notch leads sideward into the body for centering and seating the tubing in a press-fit manner. The butterfly anchor also has hinges on opposing sides of the body that support a pair of opposing foldable wings. A locking post protrudes from the distal end of one wing and when the wings are folded together the locking post may be inserted into a hole at the distal tip of the other wing for releasably locking the wings together. Thus, the wings can be conveniently folded over the catheter and latched in place to protect the user from pricking after use. On the other hand, when the wings are unfolded for use they lay perfectly flat against a patient's skin, thereby allowing adhesion of a strip of dermal tape such as Tegaderm™ overtop to anchor the catheter in place and promote needle stability.

### BRIEF DESCRIPTION OF THE DRAWINGS

Other objects, features, and advantages of the present invention will become more apparent from the following detailed description of the preferred embodiment and certain modifications thereof when taken together with the accompanying drawings in which:

FIG. 1 is a bottom perspective view of the infusion set 1 with winged anchor 2.

FIG. 2 is a top perspective view of the winged anchor 2 of FIG. 1.

FIG. 3 is a bottom view of the winged anchor 2 with catheter 6 removed.

FIG. 4 is a side view of the winged anchor 2.

### DETAILED DESCRIPTION OF THE PREFERRED EMBODIMENTS

The present invention is a subcutaneous infusion set with a winged anchor in which the wings fold over the needle and latch in place to protect it after use, and yet the wings when unfolded lie flat against the patient's skin to allow a Tegaderm™ or other dermal adhering strip to anchor the wings to the skin to promote needle stability.

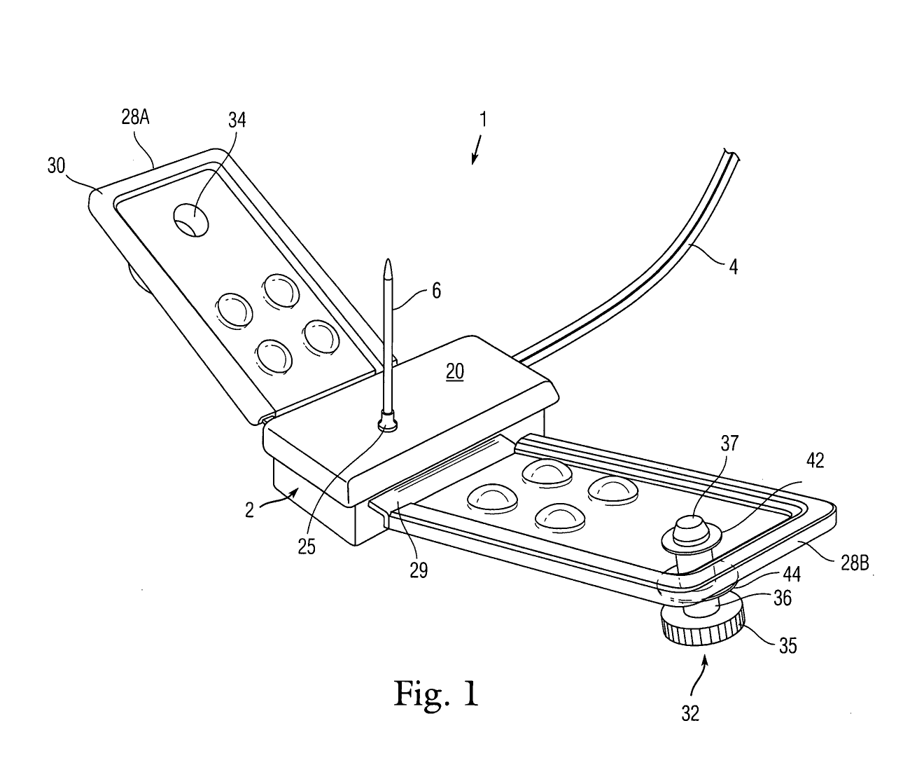

FIG. 1 is a bottom perspective view of the assembled subcutaneous infusion set 1 with winged anchor 2. The subcutaneous infusion set 1 according to the embodiment comprises flexible delivery tube 4 in fluid communication with a catheter 6, and the winged anchor 2. The winged anchor 2 generally comprises a substantially rectangular body 20 formed flat on the bottom side (illustrated) and interrupted by a hole 25 positioned toward the middle and offset slightly to the front. The catheter 6 is preferably a 90 degree catheter (formed with a right angle along its length). The flexible delivery tube 4 is in fluid communication with the catheter 6 at one end, and both the flexible delivery tube 4 and catheter 6 are friction fit within the body 20 with the angled end of the catheter 6 protruding outwardly from hole 25.

Opposing wings 28A & 28B are integrally joined to the sides of the body 20 at hinges 29 and extend outward there from. The hinges 29 are pliant plastic struts joining wings 28A & 28B to the sides of the body 20. The hinges 29 (and wings 28A & 28B) may be integrally molded, and the hinges 29 may be scored, perforated or otherwise formed with reduced thickness score-lines running lengthwise to increase the pliancy to allow easier folding at the hinges 29.

In their unfolded position, the wings 28A & 28B extend laterally on opposing sides of the body 20 and lie flat, where they can be anchored to the skin of a patient by use of suitable dermal tape such as Tegaderm™. When it is desirable to remove the infusion set 1 the wings 28A & 28B can be folded inward overtop the protruding catheter 6 until substantially adjacent to each other, fully enclosing the catheter 6 to protect against inadvertent user contact. Moreover, whilst in the folded position the wings 28A & 28B releasably latch together. This is accomplished with a detent post 32 slidably carried in one of the wings 28B and that may be press-inserted into a receiving aperture 34 formed in the other wing 28A. The detent post 32 further comprises A head 35 adapted for thumb depression, the head 35 being joined to a narrower stem 36 that passes through the wing 28B, and a distal locking tip 37 formed in a mushroom configuration for releasably locking into the receiving aperture 34 formed in the other wing 28A. The stem 36 of detent post 32 is carried in a circular aperture 42 through wing 28B and positioned toward the outer tip, with the head 35 facing the topside. The topside of wing 28B is formed with a collar 44 about the aperture 42, the collar 44 conforming to the dimensions of the head 35 in order to seat the head 35 flush thereon. Thus, with the wings 28A & 28B folded together the user need only thumb-depress the detent post 32 forcing the locking tip 37 into the receiving aperture 34 formed in the other wing 28A. This couples the two wings 28A & 28B together around the catheter 6 and prevents inadvertent exposure.

The margins of the wings 28A & 28B are formed with raised ribs 30 for structural reinforcement.

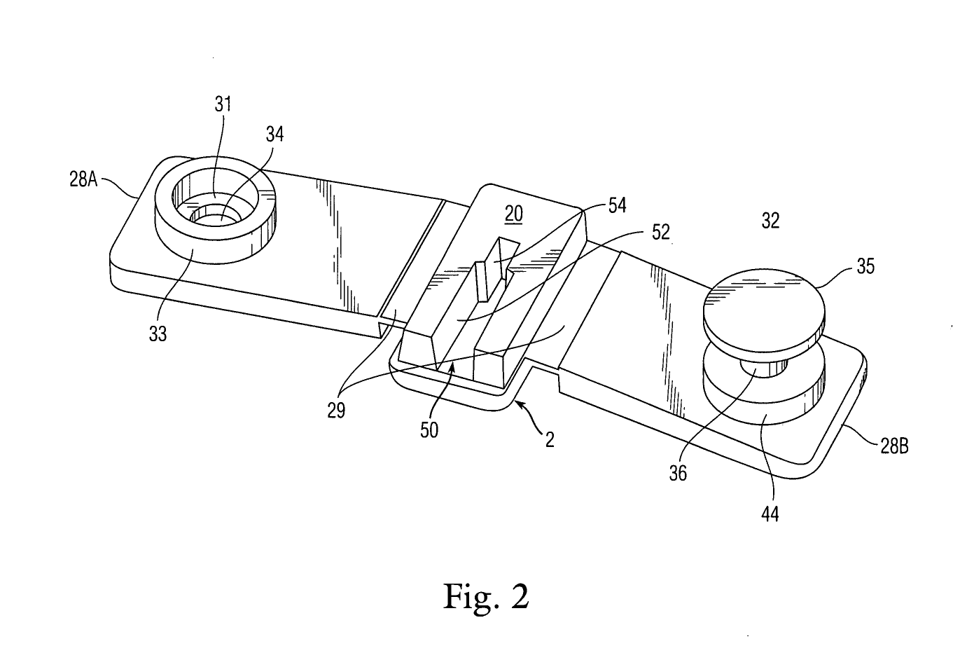

FIG. 2 is a top perspective view of the winged anchor 2 of FIG. 1 illustrating the details of body 20. In accordance with the present invention, the top of the body 20 is formed with a recessed notch 50 entering inwardly from one end of body 20 and continuing part way along the length of body 20. The notch 50 is defined by stepped sidewalls including sidewalls 52 dimensioned for press-fit insertion of the tubing 4, and constricted sidewalls 54 dimensioned for press-fit insertion of the catheter 6. The notch 50 runs to the hole 25 from which the catheter 6 exits, e.g., approximately three-fourths the length of the body 20. The notch 50 may be integrally molded into the body 20, and the press-fit conformity to the delivery tube 4 and catheter 6 allow these components to be pressed down into the notch 50 to anchor them in place, or just as easily removed. The flexible delivery tube 4 will enter the winged anchor 2 through the notch 50 in body 20, run to the center, and couple to the catheter 6 which angles and protrudes outward through the hole 25 (obscured in FIG. 2) for a 90 degree transdermal insertion.

Note the receiving aperture 34 formed in the wing 28A defined a plateau 31 that catches the locking tip 37 of detent post 32 once inserted there through to prevent backing up. In addition, wing 28A is also formed with a collar 33 circumscribing the aperture 34 to enclose the locking tip 37 of detent post 32 once inserted there through to prevent inadvertent dislodgement.

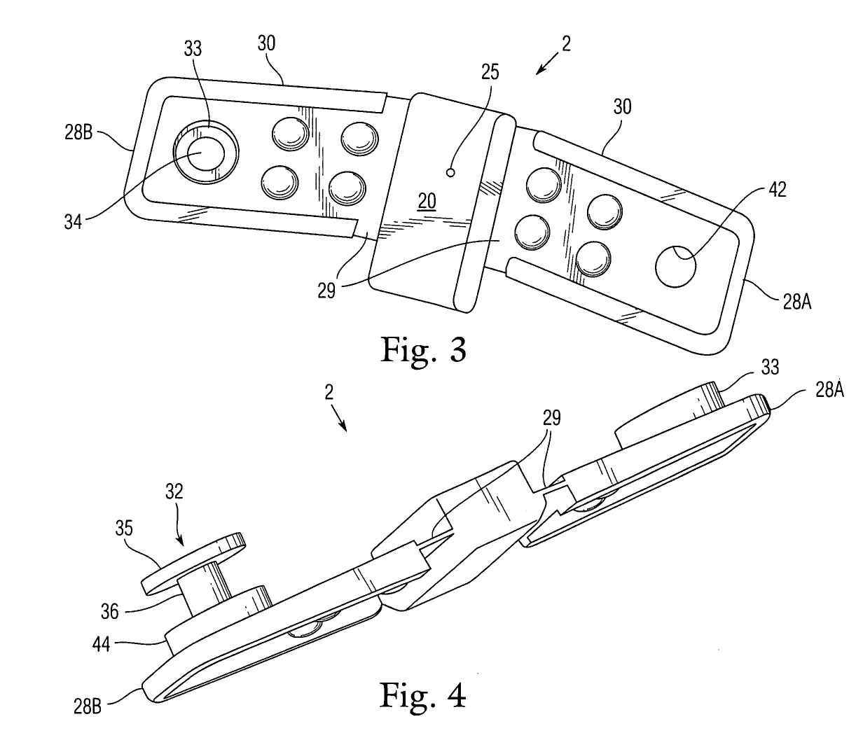

FIG. 3 is a bottom view of the winged anchor 2 with catheter 6 removed, and FIG. 4 is a side view. The length of the wings 28A, 28B is approximately 0.5 mm. and the thickness is approximately 0.06 mm. The width of the body 20 is approximately 0.29 mm. and the depth of the notch 50 is approximately 0.15 mm. The length of the entire winged anchor 2 is approximately 1.65 mm.

It should now be apparent that the present invention provides a safety subcutaneous infusion set that is easy and safe to use, with wings 28A & 28B that fold over the catheter 6 and positively (yet removably) latch in place to prevent inadvertent pricking after use. To unfurl the wings 28A, 28B a conscious effort must be made to pry the wings 28A, 28B apart and uncatch the detent post 34. Moreover, the safety subcutaneous infusion set lies perfectly flat against a patient's skin while unfolded and in use, thereby allowing adhesion of a strip of dermal tape such as Tegaderm™ overtop the wings to anchor the catheter in place and promote needle stability. Furthermore, the winged anchor 2 may be economically manufactured since it can be entirely molded from two molds, one for the winged anchor 2 and one for the detent post 34.

The winged anchors 2 of the present invention may optionally be color coded to simplify patient care and maintain patient safety by allowing a patient to easily identify problems with needle placements, occlusions, or painful swellings by simply stating to a health care professional that a certain colored winged anchor 2 poses a problem. This is helpful to a patient who will not visit with his healthcare professional until hours after injection, at which time the patient's memory may have slightly faded. Also, the colored winged anchors 2 are especially useful to a child patient. For example, a child who is having a drug delivered via four different needle sites may say to his nurse that the needle with the yellow winged anchor 2 hurts. The nurse can then easily find the problem site by searching for the yellow one.

Additionally, the colored winged anchors 2 allow a patient or health care professional to easily identify and communicate defective equipment to the equipment manufacturer. For example, needles and/or tubing may be blocked or leaking. Patients and healthcare professionals may easily identify and communicate the defective equipment to each other or to equipment manufacturers, by identifying the colored winged anchor 2 that correspond to the defective equipment.

Having now fully set forth the preferred embodiments and certain modifications of the concept underlying the present invention, various other embodiments as well as certain variations and modifications thereto may obviously occur to those skilled in the art upon becoming familiar with the underlying concept. It is to be understood, therefore, that the invention may be practiced otherwise than as specifically set forth herein.

I claim:

1. An infusion set comprising:
an angled catheter;
a length of flexible tubing in fluid communication with said catheter;
an anchoring member, said anchoring member further comprising a central body formed with a flat bottom surface, a top and sides, and a notched recess entering from the side and open at the top of said body for centering and seating the delivery tube in a press-fit manner, said notched recess leading to a through hole through the bottom surface for insertion of said angled catheter therein, said anchoring member further comprising a pair of opposing foldable wings joined to the sides of said central body at hinges, and a detent post mounted distally in one of said wings for locking engagement in an aperture positioned distally in the other said wing;
wherein when a length of said catheter protruding from said through hole is transdermally inserted into a patient the opposing wings lie flat against said patient's skin in an unfolded position to facilitate tape-adhesion of the infusion set to said skin, and when said infusion set is removed the wings may be folded together over said protruding catheter and locked together by insertion of said detent post into said aperture.

2. An infusion set according to claim 1, wherein said hinges comprise pliant struts of plastic.

3. An infusion set according to claim 2, wherein said pliant struts of plastic are scored to increase their pliancy.

4. An infusion set according to claim 1, wherein said notched recess in said body comprises a first length for centering and seating the delivery tube in a press-fit manner, and a second constricted length for centering and seating the catheter in a press-fit manner.

5. An infusion set according to claim 1, further comprising dermal tape to anchor said wings of said anchoring member to a patient's skin, when said wings are unlocked.

6. An infusion set according to claim 1, wherein said detent post is slidably carried in one of said wings.

7. An infusion set according to claim 6, wherein said detent post comprises a head for thumb depression, a stem slidably carried in one of said wings, and a distal locking tip.

8. An infusion set according to claim 1, wherein said anchoring member is color-coded for aiding the patient in easily identification.

9. An infusion set comprising:
a catheter;
a length of flexible tubing in fluid communication with said catheter;
an anchoring member, said anchoring member further comprising a substantially rectangular body formed with an elongate duct partially traversing said body for securing said flexible tube therein in a press-fit manner, said duct leading to a hole for passing said catheter, and said anchoring member further comprises hinges on opposing sides of said duct and defining distal foldable wings, the distal margin of one of said wings including a detent post and the distal margin of another of said wings including an aperture for receiving said detent post for releasably locking said foldable wings together;
whereby said wings can be conveniently folded over the catheter and latched in place to protect the user after use.

10. An infusion set according to claim 9, wherein said hinges comprise pliant struts of plastic.

11. An infusion set according to claim 10, wherein said pliant struts of plastic are scored to increase their pliancy.

12. An infusion set according to claim 9, wherein said duct in said body comprises a first length for centering and seating the delivery tube in a press-fit manner, and a second constricted length for centering and seating the catheter in a press-fit manner.

13. An infusion set according to claim 9, further comprising dermal tape to anchor said wings of said anchoring member to a patient's skin, when said wings are unlocked.

14. An infusion set according to claim 9, wherein said detent post is slidably carried in one of said wings.

15. An infusion set according to claim 14, wherein said detent post comprises a head for thumb depression, a stem slidably carried in one of said wings, and a distal locking tip.

16. An infusion set according to claim 9, wherein said anchoring member is color-coded for aiding the patient in easily identification.

17. A catheter anchoring member comprising:
a central body having a notch formed along a top side for centering and seating a delivery tube in a press-fit manner, said notch leading to a through-hole for insertion of a catheter; and
a pair of opposing foldable wings attached to said central body at hinges; and
locking means positioned distally on said pair of opposing wings for releasably locking the wings together.
